(19) United States
(12) Patent Application Publication
(10) Pub. No.: US 2011/0144699 A1
(43) Pub. Date: Jun. 16, 2011

(54) Bone Implants with Integrated Line Locks

(75) Inventors: T. Wade Fallin, Logan, UT (US); M. Mary Sinnott, Logan, UT (US)

(73) Assignee: MEDICINELODGE, INC. DBA IMDS CO-INNOVATION, Logan, UT (US)

(21) Appl. No.: 13/028,018

(22) Filed: Feb. 15, 2011

Related U.S. Application Data
(63) Continuation of application No. 11/142,933, filed on Jun. 2, 2005, now abandoned.

(51) Int. Cl. A61B 17/80; A61F 2/30

(52) U.S. Cl. 606/286; 623/18.11

(57) ABSTRACT

A bone plate has a plurality of line locks arranged along its length. Each of the line locks has a plurality of passageways designed to receive the ends of a flexible line such that one or both of the ends can be drawn through the line lock along only one direction. The lines may encircle the bone fragments to be set to keep them in place. The line locks enable the lines to be easily tightened and re-tightened as needed. A bone prosthesis has a line lock having a plurality of passageways designed to receive one or both ends of a flexible line such that one or both ends can be drawn through the line along only one direction. The line can then be used to secure the bone prosthesis to one or more bones. The bone prosthesis may be a joint prosthesis or the like.

### CROSS-REFERENCE TO RELATED APPLICATIONS

This application is a continuation of:

U.S. application Ser. No. 11/142,933, filed Jun. 2, 2005, which carries Applicants' docket no. MLI-33, and is entitled BONE IMPLANTS WITH INTEGRATED LINE LOCKS, which is incorporated herein by reference.

The following documents are incorporated herein by reference:

U.S. application Ser. No. 10/459,375, filed Jun. 11, 2003, which carries Applicants' docket no. 13447.35, and is entitled LINE LOCK SUTURE ATTACHMENT SYSTEMS AND METHODS;

U.S. application Ser. No. 10/936,376, filed Sep. 7, 2004, which carries Applicants' docket no. MLI-15 and is entitled ADJUSTABLE LINE LOCKS AND METHODS;

U.S. application Ser. No. 10/942,275, filed Sep. 15, 2004, which carries Applicants' docket no. MLI-16 and is entitled LINE LOCK THREADING SYSTEMS AND METHODS;

U.S. application Ser. No. 11/001,866, filed Dec. 1, 2004, which carries Applicants' docket no. MLI-17 and is entitled LINE LOCK SUTURE ATTACHMENT SYSTEMS AND METHODS; and

U.S. application Ser. No. 11/125,885, filed May 10, 2005, which carries Applicants' docket no. MLI-32 and is entitled COMPACT LINE LOCKS AND METHODS.

### BACKGROUND OF THE INVENTION

1. The Field of the Invention

The present invention relates generally to bone plates and bone prostheses for repairing fractures, replacing joints, and the like, and more specifically to such implants with integrated line locks that may be used to secure the implant with respect to a flexible member such as a suture or cable.

2. The Relevant Technology

A wide variety of bone implants are currently used to perform various functions. For example, bone plates are used to hold together bone fragments to enable them to set properly. A joint implant may be used to replace one or both portions of an articulating joint, such as a shoulder, hip, knee, elbow, wrist, ankle, or spinal motion segment.

Such bone implants often utilize one or more flexible members, such as sutures, cables, wires, or the like, to secure the implant to one or more bones. For example, many known bone plates have a main body with cables wrapped around the bone fragments and secured to the main body to keep the bone fragments in place with respect to the main body. A bone prosthesis may have a stem designed to be inserted into a bone canal after removal of the head of the bone and the diseased or damaged articular surface. The bone prosthesis replaces the articular surface, and the remaining pieces of the head may be secured to the bone prosthesis through the use of sutures.

Unfortunately, the manner in which known bone implants are attached to flexible members is often quite cumbersome, particularly in a surgical environment. Many known bone implants are not compatible with MIS (Minimally Invasive Surgical) techniques that could otherwise be used to expedite healing. The flexible members may have to be crimped to the bone implant, thereby requiring insertion of the working end of a crimping device into the surgical site. Alternatively, the flexible members may need to be tied in place or secured via fasteners such as set screws. Such attachment methods may require the use of additional tooling and/or a relatively large access space in the surgical site.

Typically, known bone implants are also unable to receive the flexible members in a manner that permits easy tightening. Rather, the flexible member must be detached from the bone implant and re-secured with the desired level of tension. This can pose a significant problem for implants such as bone plates that have multiple flexible lines because tightening one flexible line may ease the tension on other, previously secured flexible lines to unacceptable levels. Accordingly, there is a need for bone implants that may be easily secured to flexible lines in a manner that is simple, easily re-tightenable, and compatible with MIS techniques.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is an exploded, perspective view of a bone implant according to one embodiment of the invention, comprising a bone plate incorporating three line locks.

FIG. 2 is a perspective view of the bone implant of FIG. 1, in use to secure two bone fragments together via three flexible members passing around the bone fragments and secured to the line locks.

FIG. 3 is a perspective view of a bone implant according to one alternative embodiment of the invention, comprising a bone plate in use to secure two bone fragments together, wherein the bone plate has three differently configured line locks.

FIG. 4 is a perspective view of a bone implant according to another alternative embodiment of the invention, comprising a humeral prosthesis incorporating a line lock.

FIG. 5 is a perspective view of the bone implant of FIG. 4, with two flexible member ends secured to the line lock to enable attachment of the bone implant to bone.

### DETAILED DESCRIPTION OF THE PREFERRED EMBODIMENTS

The present invention relates to line locks that can be used in part to replace surgical knots tied in sutures in open, arthroscopic, and endoscopic procedures. By increasing the size of the line locks, it is also appreciated that the line locks can be used outside of surgical procedures for any use where it is desired to selectively adjust and/or tie off a line such as a rope, cord, string, or other conventional type of line.

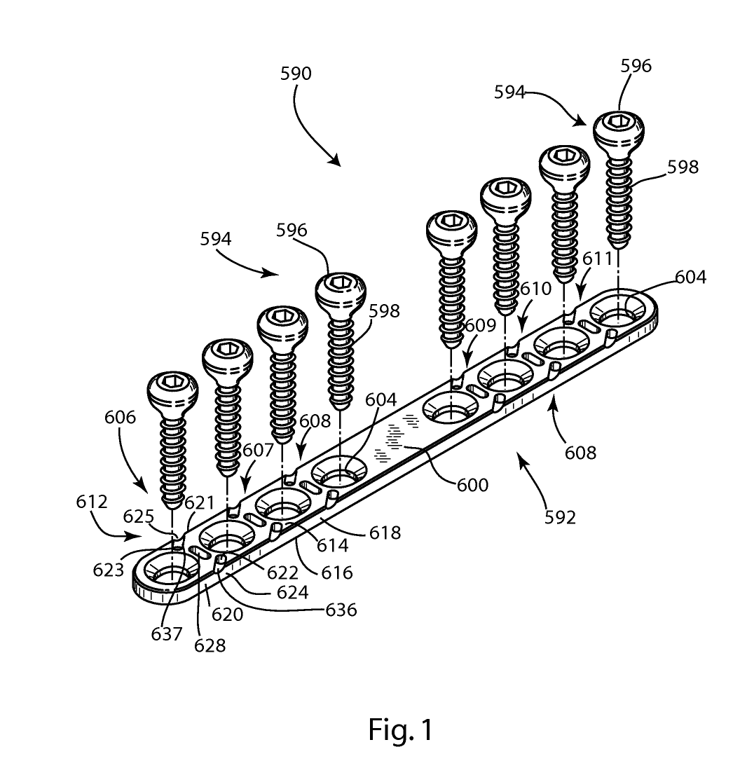

According to the present invention, a line lock may be incorporated in a wide variety of orthopedic devices that are to be secured to a suture, wire, cable, or other line. For example, as will be shown and described in connection with FIGS. 1 through 5, bone implants such as bone plates and joint implants may incorporate line locks to facilitate attachment and tensioning of flexible lines.

In this application, the term “couple” broadly refers to connection of two items to each other. Two items may be “coupled” if they are connected together in a manner that prevents relative motion on one direction, but not another. A “longitudinal length” of an object is the length of the object along its longest dimension. “Cooperation” of a plurality of passageways to receive multiple suture portions does not require that each suture portion pass through all of the cooperating passageways.

With reference to a line, “locking” refers to retention of a portion of the line in a manner that substantially prevents motion of the portion along at least one direction. “Active locking” refers to locking that is carried out by deliberately actuating some part of a locking mechanism to effect locking of the line, aside from moving the line itself. “Passive locking” refers to locking that does not require a user to actuate any component besides the line to effect locking. Passive locking may be carried out through the use of moving parts such as pivoting pawls, but may alternatively be effected through the geometry of various passageways of a line lock with no moving parts.

In this application, the phrase “substantially bounded aperture” refers to an aperture that is sufficiently encircled by material to prevent a line from exiting the aperture via motion transverse to the length or axis of the aperture. Thus, a substantially bounded aperture may be fully encircled by material, or may have one or more breaks, each of which is smaller than the diameter of the line that is to pass through the substantially bounded aperture.

The term “abutting” refers to two objects that contact each other, either directly or indirectly, such that one of the objects rests against the other. The two objects need not directly contact each other. For example, an implant that “abuts” bone rests against the bone, but may be separated from the bone by tissue such as a layer of fascia. The term “bone” need not be a whole, natural bone, but may instead be a bone fragment. The term “tissue” may be broadly interpreted to include bone tissue, ligaments, muscles, and the like.

In this application, a “bone plate” is a member used to secure two bone fragments with respect to each other to facilitate healing. A “bone prosthesis” is an implant used to replace at least a portion of a bone to carry out the function performed by the replaced portion of bone. Accordingly, bone anchors for suture attachment and similar devices that are implanted in healthy bone tissue to help heal surrounding tissues are not bone prostheses, as defined herein. A “joint prosthesis” is a bone prosthesis used to replace an articulating surface of a bone, thereby replacing at least part of the corresponding joint.

Referring to FIG. 1, an exploded, perspective view illustrates a bone implant 590 according to one embodiment of the invention. The bone implant 590 comprises a bone plate 592 designed to help secure two bone fragments (not shown in FIG. 1) together for proper healing. A plurality of screws 594 may be used to secure the bone plate 592 to the bone fragments. Each of the screws 594 may be of a type commonly used for implantation in bone. Each screw 594 may have a head 596 designed to be driven by a hexagonal driving feature, with a rounded shape that permits each screw 594 to be driven into the bone along an orientation nonperpendicular to the bone plate 592. Each screw 594 may also have a shank 598 with a threaded shape designed to be threadably implanted in the bone.

The bone plate 592 has a central portion 600, with a plurality of holes 604 arrayed along either side of the central portion 600 to receive the screws 594. In the embodiment of FIG. 1, the bone plate 592 has eight holes 604 arranged to receive eight screws 594. However, the number of screws 594 and the number of holes 604 are merely exemplary, as is the aspect ratio and overall geometry of the bone plate 592 of FIG. 1.

As also shown in FIG. 1, the bone plate 592 has a first line lock 606, a second line lock 607, a third line lock 608, a fourth line lock 609, a fifth line lock 610, and a sixth line lock 611. Each of the line locks 606, 607, 608, 609, 610, 611 is positioned between a pair of adjacent holes 604. Each of the line locks 606, 607, 608, 609, 610, 611 has a body 612 with a top surface 614, a bottom surface 616, and a periphery 618, which are numbered only in connection with the first line lock 606 for clarity. In the embodiment of FIG. 1, the bodies 612 of all of the line locks 606, 607, 608, 609, 610, 611 are unitarily interconnected to define the shape of the bone plate 592.

Each of the line locks 606, 607, 608, 609, 610, 611 has a first end 620 and a second end 621. The body 612 of each of the line locks 606, 607, 608, 609, 610, 611 is shaped to define a first primary passageway 622, a second primary passageway 623, a first secondary passageway 624, a second secondary passageway 625, and a first working passageway 628. The first and second primary passageways 622, 623 and the first working passageway 628 are all fully bounded. The first and second secondary passageways 624, 625 are each only partially bounded. The secondary passageways 624, 625 are contiguous with the periphery 618 because the bore of each of the secondary passageways 624, 625 transitions directly into the periphery 618, with no significant intervening surface.

The first and second primary passageways 622, 623 are each generally circular in shape. The first working passageway 628 is designed to accommodate both locking portions 356, 357 of a suture 350 (not shown in FIG. 1), and is thus elongated in shape. The first working passageway 628 is positioned between the first and second primary passageways 622, 623 such that the passageways 622, 623, 628 are arrayed in a generally straight line. The first working passageway 628 has an elongated shape designed to receive multiple suture portions. The passageways 622, 623, 624, 625, 628 are radially symmetrical to each other.

The body 612 of each of the line locks 606, 607, 608, 609, 610, 611 also defines a first groove 636 and a second groove 637, both of which are formed in the top surface 614. The first groove 636 extends along a generally straight path between the first primary and secondary passageways 622, 624. Similarly, the second groove 637 extends along a generally straight path between the second primary and secondary passageways 623, 625. The first and second grooves 636, 637 serve to enhance suture locking by each of the line locks 606, 607, 608, 609, 610, 611 by forming bends in the working portions 354, 355 when the working portions 354, 355 are pressed into them.

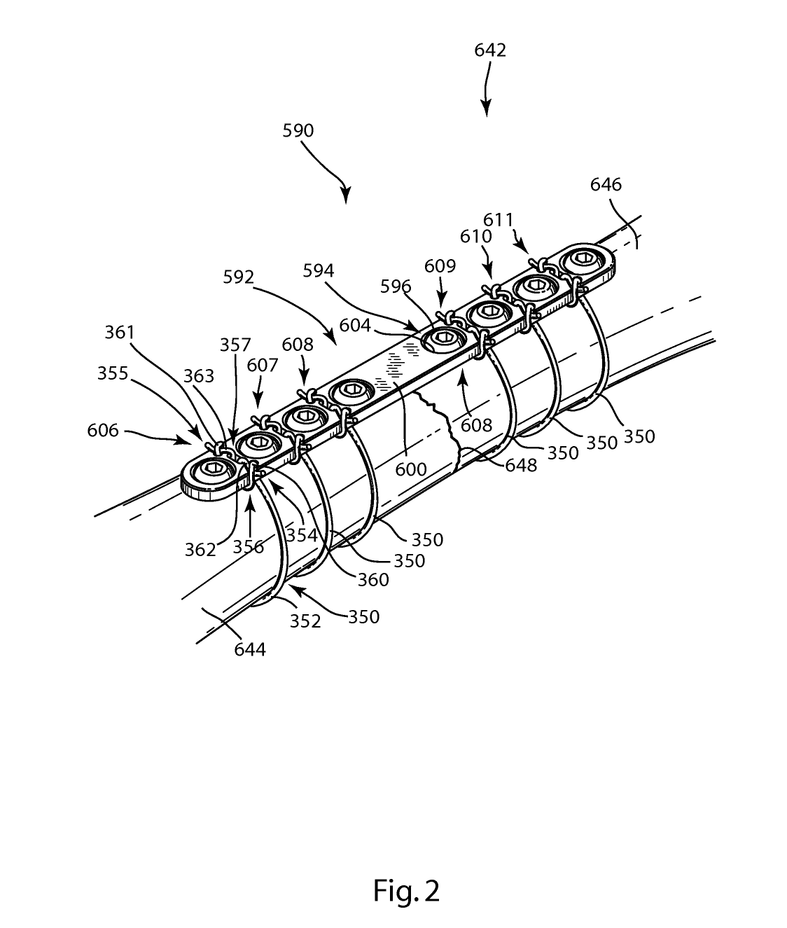

Referring to FIG. 2, a perspective view illustrates the bone implant 590 of FIG. 1, with a suture 350 routed tightly through the passageways 622, 623, 624, 625, 628 of each of the line locks 606, 607, 608, 609, 610, 611 of the bone plate 592. The bone implant 590 and sutures 350 define a system 642 for securing a first bone fragment 644 to a second bone fragment 646. The first bone fragment 644 and the second bone fragment 646 are pieces of a single bone separated by a fracture 648. The system 642 may be used to promote proper setting and healing of the first and second bone fragments 644, 646 to eliminate the fracture 648. The screws 594 may optionally be used to secure the bone plate 592 to the first and second bone fragments 644, 646 prior to securement via the sutures 350. Alternatively, the sutures 350 may be applied prior to the screws 594.

The sutures 350 may be routed through the passageways 622, 623, 624, 625, 628 of each corresponding line lock 606, 607, 608, 609, 610, 611 in the same manner. The locking portions 356, 357 of each suture 350 pass upward through the corresponding first and second primary passageways 622, 623, respectively, and then pass parallel to the first and second grooves 636, 637 to define the first and second compression sections 360, 361. The first and second locking portions 356, 357 then pass through the first and second secondary passageways 624, 625, respectively. From the first and second secondary passageways 624, 625, the locking portions 356, 357 pass underneath the bone plate 592 and upward, through the first working passageway 628. From the working passageway 628, the first compressed section 362 of the first working portion 354 extends between the first compression section 360 and the first groove 636, and the second compressed section 363 of the second working portion 355 extends between the second compression section 361 and the second groove 637.

Thus, the first locking portion 356 of each suture 350 passes through the corresponding line lock 606, 607, 608, 609, 610, 611 along a first pathway. The second locking portion 357 of each suture 350 passes through the corresponding line lock 606, 607, 608, 609, 610, 611 along a second pathway.

When the sutures 350 are routed as shown, the line locks 606, 607, 608, 609, 610, 611 provide passive locking that resists elongation of a standing portion 352 of the suture 350. As the standing portion 352 of each suture 350 is tightened, tension is exerted on the corresponding compression sections 360, 361. The compression sections 360, 361 then press the compressed sections 362, 363, respectively, against the top surface 614 to cause the compressed sections 362, 363 to frictionally engage the grooves 636, 637, respectively. Working portions 354, 355 of the suture 350 form bends where they extend across the grooves 636, 637 to enhance locking by adding to the frictional resistance to motion of the working portions 354, 355.

The locking provided by the line locks 606, 607, 608, 609, 610, 611 may be referred to as “passive locking” because no part of the line locks 606, 607, 608, 609, 610, 611 need be actuated to effect locking, independently of motion of the suture 350. Rather, the surgeon need only move the suture 350 to cause locking to occur. Furthermore, the line locks 606, 607, 608, 609, 610, 611 carry out locking without the use of moving parts.

The sutures 350 may be inserted into the passageways 622, 623, 624, 625, 628, passed around the corresponding bone fragment 644 or 646, tightened, and locked within the line locks 606, 607, 608, 609, 610, 611. If desired, a pair of threaders (not shown) may be routed through the passageways 622, 623, 624, 625, 628 of each of the line locks 606, 607, 608, 609, 610, 611 to facilitate routing of the sutures 350. Each threader may have a leading end with a handle or other pull feature, and a trailing end with a loop through which the suture end may be inserted and retained. The threaders may then be drawn through the passageways 622, 623, 624, 625, 628 to draw locking portions 356, 357 of the sutures 350 through the passageways 622, 623, 624, 625, 628 along the first and second pathways.

As described in connection with the previous embodiment, the working portions 354, 355 may be cut short after the suture 350 has been tightened and locked by the line lock 410. The line lock 410 may also be formed of a variety of bioabsorbable or non-bioabsorbable materials.

Advantageously, the sutures 350 can easily be tensioned and re-tensioned. Thus, the surgeon has a great deal of flexibility regarding the order in which the sutures 350 are positioned and tensioned. Once one of the sutures 350 is routed through one of the line locks 606, 607, 608, 609, 610, 611 and tensioned, re-tensioning can be carried out by simply drawing the working portions 354, 355 of the suture 350 to draw the locking portions 356, 357 further along their pathways through the corresponding passageways 622, 623, 624, 625, 628. The suture 350 need not be unlocked by any separate operation prior to re-tensioning. This is advantageous because often, as multiple flexible members are used to secure a bone plate to bone fragments, the flexible members that are tensioned first will loosen when the other flexible members are tightened. Without the aid of the present invention, tightening the lines on the entire bone plate may require several tensioning and re-tensioning steps.

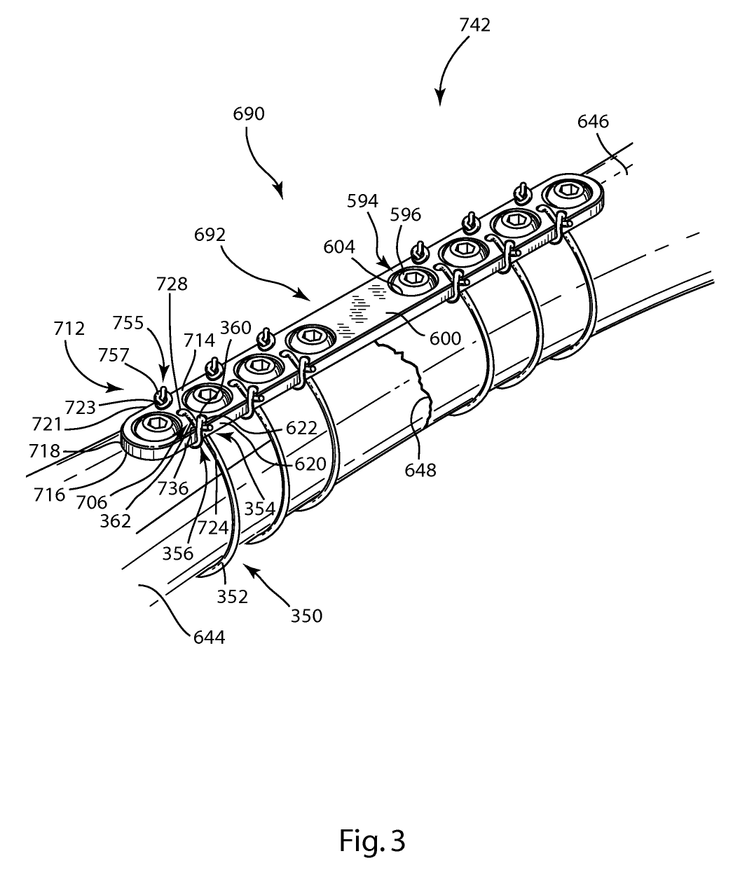

FIGS. 1 and 2 represent only one of many embodiments of the invention in which one or more line locks are incorporated into a bone plate. According to some alternative embodiments, different line lock configurations may be used. For example, line locks according to the invention need not permit both suture ends to be drawn through the line lock for tensioning; rather, one end of the suture may be pre-attached to the line lock. One example of such an embodiment will be set forth in connection with FIG. 3.

Referring to FIG. 3, a perspective view illustrates a bone implant 690 according to one alternative embodiment of the invention. The implant 690 includes a bone plate 692 and screws 594 like those of the previous embodiment. The bone plate 692 has a shape similar to that of the bone plate 694 of the previous embodiment, with a central portion 600 and a plurality of holes 604 arrayed in a generally linear fashion on either side of the central portion 600 to receive the screws 594. The bone plate 692 has a first line lock 706, a second line lock 707, a third line lock 708, a fourth line lock 709, a fifth line lock 710, and a sixth line lock 711, each of which is positioned between an adjacent pair of the holes 604.

The line locks 706, 707, 708, 709, 710, 711 are configured similarly to the line locks 606, 607, 608, 609, 610, 611 of the previous embodiment. More precisely, each of the line locks 706, 707, 708, 709, 710, 711 has a body 712 with a top surface 714, a bottom surface 716, and a periphery 718. The body 712 of each of the line locks 706, 707, 708, 709, 710, 711 has a first end 620 and a second end 721. A first primary passageway 622, a retention passageway 723, a first secondary passageway 624 a first working passageway 728, and a first groove 636 are formed in the body 712. The first primary passageway 622, the first secondary passageway 624, and the first groove 636 are like those of the previous embodiment, but the retention passageway 723 has no adjoining groove, and the first working passageway 728 is circular and is sized to receive only one suture portion.

The bone implant 690, in combination with the sutures 350, forms a system 742 that may be used to retain the first and second bone fragments 644, 646 in a manner somewhat similar to that of the previous embodiment. The screws 594 may be applied to secure the bone plate 692 to the bone fragments 644, 646 prior to or after attachment of the sutures 350. The first locking portion 356 of each suture 350 is routed through the first primary passageway 622, the first secondary passageway 624, and the first working passageway 628 in same manner as in the previous embodiment. As in the previous embodiment, the first groove 636 enhances locking of the first locking portion 356, and the first locking portion 356 is automatically locked to prevent motion of the first locking portion 356 through the first end 620 in a direction that would permit elongation of the standing portion 352.

However, in place of a second locking portion that is independently tensionable and lockable, the suture 350 of the system 742 is routed through the retention passageway 723 and secured to provide a secured end 755. The secured end 755 is attached to the second end 721 in such a manner that it is locked in place at a single position that cannot be adjusted, at least with respect to motion in a direction that permits elongation of the standing portion 352. In FIG. 3, a knot 757 is formed in the secured end. The knot 757 is larger than the retention passageway 723, and therefore prevents the suture 350 from moving through the retention passageway 723 along one direction. Alternative embodiments may utilize an attachment mechanism that prevents motion of one end of the suture 350 with respect to the second end 721 along any direction. For example, the end of the suture 350 may be bonded to the second end 721 with an adhesive, secured with a fastener, or even insert molded within the interior of the second end 721.

In operation, the system 742 is similar to the system 642. However, only the first working portion 354 of each suture 350 need be drawn to tension the sutures 350. If desired, the sutures 350 may be secured to the second ends 721 prior to delivery to the surgeon, so that the surgeon need only route the first locking portion 356 through the first primary, secondary, and working passageways 622, 624, 728 after passage of the sutures 350 around the first and second bone fragments 644, 646. As in the previous embodiment, threaders or other devices may be used to facilitate insertion of the first locking portions 356 through the passageways 622, 624, 728.

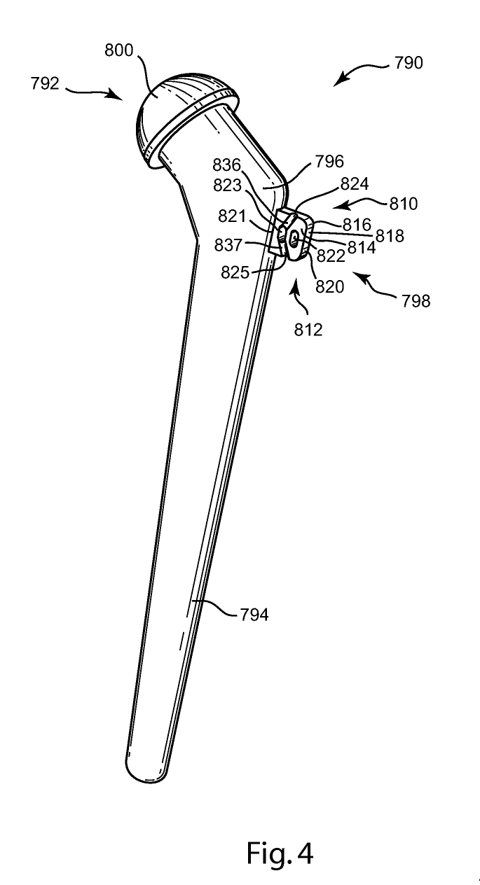

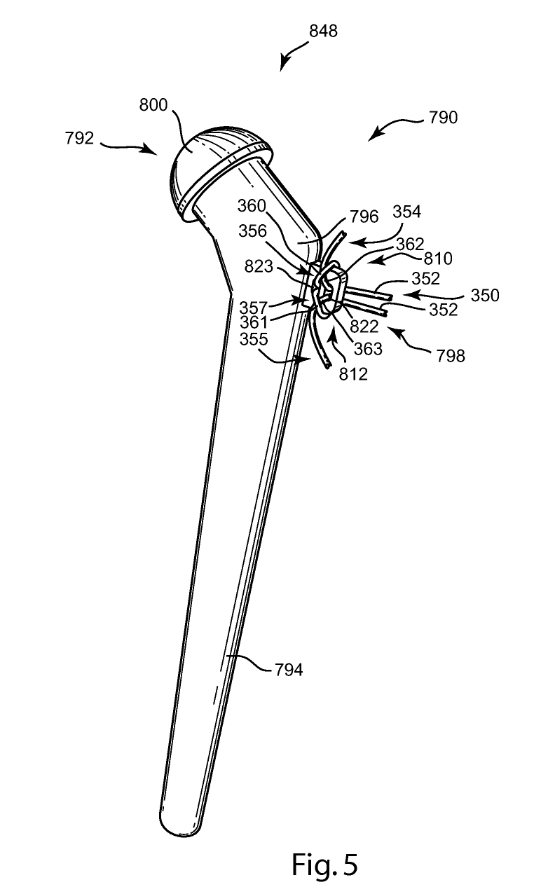

Line locks may be incorporated into a wide variety of bone implants besides bone plates. For example, line locks may also be useful for a wide variety of joint prostheses, including, but not limited to a femoral component of a hip prosthesis, an acetabular component of a hip prosthesis, a femoral component of a knee prosthesis, a tibial component of a knee prosthesis, a humeral component of a shoulder prosthesis, a glenoid component of a shoulder prosthesis, a humeral component of an elbow prosthesis, an ulnar component of an elbow prosthesis, a radial component of a wrist prosthesis, a carpal component of a wrist prosthesis, a tibial component of an ankle prosthesis, a tarsal component of an ankle prosthesis, a total disc replacement, and a facet joint replacement. Many such joint prostheses are secured to flexible members such as sutures to help attach them to the corresponding bones, or to reattach removed bone fragments. One example of a joint prosthesis that incorporates a line lock will be shown and described in connection with FIGS. 4 and 5.

Referring to FIG. 4, a perspective view illustrates a bone implant 790, which may be a humeral component of a shoulder prosthesis. The bone implant 790 has a head 792, a stem 794, and a bend 796 that provides a predetermined angle of the head 792 with respect to the stem 794. The bone implant 790 also has a flange 798 that extends generally perpendicular to the stem 794. The head 792 has an articular surface 800 shaped to articulate with the socket of the shoulder to replace the natural semispherical articulating surface of the humerus. The stem 794 is designed to be inserted into the interior canal of the humerus. The flange 798 helps to prevent rotation of the stem 794 within the bone, and also provides a line lock 810 that facilitates attachment of a flexible member, such as a suture (not shown in FIG. 4) to the bone implant 790.

As shown, the line lock 810 has a body 812 with a top surface 814, a bottom surface 816, and a periphery 818. The body 820 also has a first end 820 and a second end 821. The body 820 defines a first primary passageway 822, a first secondary passageway 824, a second secondary passageway 825, and a first working passageway 828. The first primary passageway 822 and the first working passageway 828 are elongated so that each of them is able to receive two suture portions. The first primary and working passageways 822, 828 are both fully bounded by the body 812, but the first and second secondary passageways 824, 825 are only partially bounded. The body 812 also defines first and second grooves 836, 837 that extend between the first primary passageway 822 and the first and second secondary passageways 824, 825, respectively.

Referring to FIG. 5, a perspective view illustrates a system 848 including the bone implant 790 of FIG. 4 and a suture 350. As in previous embodiments, the suture 350 has a standing portion 352, first and second working portions 354, 355, and first and second locking portions 356, 357. The first and second locking portions 356, 357 are routed through the passageways 822, 824, 825 in a symmetrical manner. As shown, the locking portions 356, 357 may pass underneath the body 812 (with respect to the view of FIG. 5), and up through the first primary passageway 822. From the first primary passageway 822, the first and second locking portions 356, 357 extend generally parallel to the first and second grooves 836, 837, and through the first and second secondary passageways 824, 825 to define first and second compression sections 360, 361, of the first and locking portions 356, 357, respectively. From the secondary passageways 824, 825, the first and second locking portions 356, 357 extend underneath the line lock 810 and through the first working passageway 828.

From the first working passageway 828, the first and second working portions 354, 355 extend between the first and second compression sections 360, 361, and the first and second grooves 836, 837, to define first and second compressed sections 362, 363, of the first and second working portions 354, 355, respectively. As in previous embodiments, tension on the working portions 354, 355 enables the locking portions 356, 357 to advance through the passageways 822, 824, 825, 828 to shorten the working portion 352. Tension on the standing portion 352 causes the compression sections 360, 361 to press the compressed sections 362, 363 into the grooves 836, 837 to lock the locking portions 356, 357, thereby preventing elongation of the standing portion 352.

In use, the surgeon may remove the head of the humerus (not shown), including the natural articular surface, and may form a hole through the interior canal of the humerus. The surgeon may then insert the stem 794 into the hole and align the bone implant 790 such that the articular surface 800 of the head 792 approximates the original position and orientation of the natural articular surface. Then, the surgeon may encircle one or more bone fragments of the natural humeral head with the standing portion 352 of the suture 350 and insert the locking portions 356, 357 of the suture 350 through the passageways 822, 824, 825, 828. As in previous embodiments, threaders or other devices may be used to facilitate routing of the locking portions 356, 357.

The one or more bone fragments may then be positioned as desired, for example, near their original positions, and then the working portions 354, 355 of the suture 350 may be drawn to tighten the standing portion 352 to hold the one or more bone fragments in place. The suture 350 facilitates incorporation of the bone fragments back into the natural humerus.

According to one alternative embodiment (not shown), multiple line locks may be provided to enable multiple sutures 350 to be easily secured to a joint prosthesis. Furthermore, one or more line locks configured differently from the line lock 810 of FIGS. 4 and 5 may be used to provide different suture routing patterns, locking characteristics, attachment positions, and the like. Those of skill in the art will recognize the manner in which line locks may easily be incorporated into other types of bone implants, including other joint prostheses.

The present invention may be embodied in other specific forms without departing from its spirit or essential characteristics. It is appreciated that various features of the systems and methods described above can be mixed and matched to form a variety of other alternatives. As such the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for securing a bone and a tissue together, the system comprising:
a first flexible member comprising a first locking portion; and
an implant selected from the group consisting of a bone prosthesis and a bone plate, the implant comprising:
a bone engagement portion comprising a surface shaped to abut the bone; and
a first line lock portion comprising a plurality of passageways configured to receive the first locking portion of the first flexible member;
wherein the plurality of passageways are arranged such that, in response to routing of the first flexible member through the passageways along a pathway defined by the routing of the flexible member, the first locking portion is able to be drawn through the first line lock portion along only a first direction.

2. The system of claim 1, wherein the implant comprises a bone prosthesis, wherein the tissue is selected from the group consisting of a tendon and a ligament.

3. The system of claim 1, wherein the bone prosthesis is selected from the group consisting of a femoral component of a hip prosthesis, an acetabular component of a hip prosthesis, a femoral component of a knee prosthesis, a tibial component of a knee prosthesis, a patellar component of a knee prosthesis, a humeral component of a shoulder prosthesis, a glenoid) component of a shoulder prosthesis, a humeral component of an elbow prosthesis, an ulnar component of an elbow prosthesis, a radial component of a wrist prosthesis, a carpal component of a wrist prosthesis, a tibial component of an ankle prosthesis, a tarsal component of an ankle prosthesis, an endplate component of a total disc replacement, and an articular component of a facet joint replacement.

4. The system of claim 1, wherein the implant further comprises a tissue engagement portion shaped to abut the tissue.

5. The system of claim 1, wherein the first flexible member comprises a cable formed of metal.

6. The system of claim 1, wherein the first flexible member is formed of polymer.

7. The system of claim 1, wherein the first line lock portion is further configured to receive a second locking portion of the first flexible member such that the second locking portion is able to be drawn through the first line lock portion substantially along only a second direction.

8. The system of claim 7, wherein the first line lock portion comprises first, second, and third substantially bounded passageways, wherein the second substantially bounded passageway is between the first and third substantially bounded passageways and is shaped to receive both the first locking portion and the second locking portion.

9. The system of claim 1, wherein the implant further comprises a second line lock portion configured to receive a second locking portion of a second flexible member such that the second locking portion is able to be drawn through the second line lock portion substantially along only a second direction, wherein the second line lock portion is spaced apart from the first line lock portion.

10. The system of claim 1, wherein the plurality of passageways comprises two substantially bounded passageways arranged to receive the first locking portion such that, in response to tension tending to draw the first locking portion against the first direction, a compression section of the first locking portion presses a compressed section of the first locking portion against the first line lock portion.

11. The system of claim 1, wherein the first line lock portion is configured to lock a position of the first locking portion with respect to the first line lock portion independently of motion of any component, aside from the first flexible member, with respect to the first line lock portion.

12. The system of claim 1, wherein the bone engagement portion is shaped to abut an exterior surface of the bone.

13. The system of claim 1, wherein the bone engagement portion is shaped to be embedded within the bone to abut an interior surface of the bone.

14. The system of claim 1, wherein the implant comprises a bone plate, wherein the tissue comprises a second bone.

15. A system for securing a bone and a tissue together, the system comprising:
an implant selected from the group consisting of a bone prosthesis and a bone plate, the implant comprising:
a bone engagement portion comprising a surface shaped to abut the bone; and
a first line lock portion comprising at least two substantially bounded passageways that cooperate to define a first pathway defined by the routing of a first flexible member and shaped such that a first locking portion of the first flexible member extending along the first pathway comprises a compression section and a compressed section, the compressed section extending between the compression section and the first line lock portion such that, in response to tension in the first locking portion, the compressed section is pressed against the first line lock portion into one of the at least two substantially bounded passageways by the compression section;
wherein the first line lock portion is further configured to receive a second locking portion of the first flexible member such that the second locking portion is able to be drawn through the first line lock portion substantially along only a second direction.

16. The system of claim 15, wherein the implant comprises a bone prosthesis, wherein the tissue is selected from the group consisting of a tendon and a ligament, wherein the bone prosthesis is selected form the group consisting of a femoral component of a hip prosthesis, an acetabular component of a hip prosthesis, a femoral component of a knee prosthesis, a tibial component of a knee prosthesis, a patellar component of a knee prosthesis, a humeral component of a shoulder prosthesis, a glenoid component of a shoulder prosthesis, a humeral component of an elbow prosthesis, an ulnar component of an elbow prosthesis, a radial component of a wrist prosthesis, a carpal component of a wrist prosthesis, a tibial component of an ankle prosthesis, a tarsal component of an ankle prosthesis, an endplate component of a total disc replacement, and an articular component of a facet joint replacement.

17. The system of claim 15, wherein the first line lock portion comprises first, second, and third substantially bounded passageways, wherein the second substantially bounded passageway is between the first and third substantially bounded passageways and is shaped to receive both the first locking portion and the second locking portion.

18. The system of claim 15, wherein the implant further comprises a second line lock portion configured to receive a second locking portion of a second flexible member such that the second locking portion is able to be drawn through the second line lock portion substantially only along a second direction, wherein the second line lock portion is spaced apart from the first line lock portion.

19. A method for securing a bone and tissue together, the method comprising:
abutting the bone with a bone engagement portion of an implant selected from the group consisting of a bone prosthesis and a bone plate;
positioning a first flexible member to secure the tissue to the implant;
passing a first locking portion of the first flexible member through the implant through a plurality of passageways of a first line lock portion of the implant, wherein the plurality of passageways defines a first pathway; and
locking a position of the first locking portion of the first flexible member with respect to the first line lock portion of the implant in response to routing the first locking portion through the first line lock portion.

20. The method of claim 19, further comprising abutting the tissue with a tissue engagement portion of the implant.

21. The method of claim 19, further comprising passing a second locking portion of the first flexible member through the implant through a plurality of passageways defining a second pathway.

22. The method of claim 21, further comprising locking a position of a second locking portion of the first flexible member with respect to the first line lock portion in response to routing the second locking portion through the plurality of passageways of the first line lock portion.

23. The method of claim 21, further comprising locking a position of a second locking portion of a second flexible member with respect to a second line lock portion of the implant in response to routing the second locking portion through a plurality of passageways of the second line lock portion, wherein the second line lock portion is spaced apart from the first line lock portion.

24. The method of claim 19, wherein locking the position of the first locking portion with respect to the first line lock portion comprises pressing a compressed section of the first locking portion against the first line lock portion with a compression section of the first locking portion.

25. The method of claim 19, wherein abutting the bone with the bone engagement portion comprises abutting an exterior surface of the bone with the bone engagement portion.

26. The method of claim 19, wherein abutting the bone with the bone engagement portion comprises embedding the bone engagement portion within the bone to abut an interior surface of the bone.
